(19) United States
(12) Patent Application Publication
(10) Pub. No.: US 2025/0127392 A1
(43) Pub. Date: Apr. 24, 2025

(54) SYSTEMS AND METHODS FOR IDENTIFYING DOUBLE VISION

(71) Applicant: Thomas Jefferson University, Philadelphia, PA (US)

(72) Inventors: James J. Evans, Newtown Square, PA (US); Nikolaos Mouchtouris, Philadelphia, PA (US); Vadim Geyfman, Marlton, NJ (US); Stephane Krumenacker, New York, NY (US)

(21) Appl. No.: 18/723,162

(22) PCT Filed: Dec. 22, 2022
(86) PCT No.: PCT/US2022/053875

§ 371 Date: Jun. 21, 2024

Related U.S. Application Data
(60) Provisional application No. 63/293,283, filed on Dec. 23, 2021.

(51) Int. Cl. A61B 3/08; A61B 3/00; G16H 15/00; G16H 50/20

(52) U.S. Cl. CPC A61B 3/08; A61B 3/0033; A61B 3/0041; G16H 15/00; G16H 50/20

(57) ABSTRACT

A computer-implemented method is provided herein. One aspect of the invention provides a computer-implemented method including: (a) displaying a marker at a location on a display screen; (b) repositioning the marker in different locations across the display screen; (c) receiving, from a user input mechanism, user input; (d) determining a given location of the marker when the user input is received; and (e) determining from the received input, that a user views double vision of the marker at the given location.

### CROSS-REFERENCE TO RELATED APPLICATION

The present application claims priority to U.S. Provisional Patent Application No. 63/293,283, filed Dec. 23, 2021, which is incorporated herein by reference in its entirety.

### BACKGROUND

Conventional medical practices are often limited to in-person meetings between a patient and a medical professional. This can be a great burden on a patient, particularly where the patient lives a significant distance away from a corresponding medical center, or if the patient's medical condition requires numerous patient-medical professional interactions.

Telemedicine offers the ability to reduce these patient burdens. However, while advances have been made in telemedicine, conventional telemedicine platforms are limited in their ability to perform certain examinations. For example, there is no conventional system capable of implementing remote testing and tracking of double vision. Double vision (diplopia) can occur due to a number of reasons that can either pertain to pathologies involving the eye or the brain. Double vision can be binocular (only present when both eyes are open) or monocular (present even when only one eye is open). People usually experience double vision when they are looking at a specific direction but sometimes it can be present at all times.

### SUMMARY

One aspect of the invention provides a computer-implemented method including: (a) displaying a marker at a location on a display screen; (b) repositioning the marker in different locations across the display screen; (c) receiving, from a user input mechanism, user input; (d) determining a given location of the marker when the user input is received; and (e) determining from the received input, that a user views double vision of the marker at the given location.

Another aspect of the invention provides a device for generating a neuro-ophthalmic examination report. The device includes a display screen. The device also includes a user input mechanism. The device also includes one or more processors configured to execute a set of instructions that cause the one or more processors to: (a) display a marker at a location on the display screen; (b) reposition the marker in different locations across the display screen; (c) receive, from the user input mechanism, user input; (d) determine a given location of the marker when the user input is received; and (e) determine, from the received input, that a user views double vision of the marker at the given location.

Another aspect of the invention provides a computer-readable medium for generating a neuro-ophthalmic examination report. The computer-readable medium includes one or more processors. The computer-readable medium also includes memory. The computer-readable medium also includes a set of instructions stored in the memory that, when executed by the one or more processors, cause the one or more processors to: (a) display, via a display screen, a marker at a location on the display screen; (b) reposition the marker in different locations across the display screen; (c) receive, from a user input mechanism, user input; (d) determine a given location of the marker when the user input is received; and (e) determine, from the received input, that a user views double vision of the marker at the given location.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a system for double vision tracking according to embodiments of the present disclosure.

FIG. 2 depicts a server for double vision tracking procedures according to embodiments of the present disclosure.

FIGS. 3-5 depict screenshots of a double vision procedure according to embodiments of the present disclosure.

FIG. 6 depicts a process flow for a double vision procedure according to embodiments of the present disclosure.

FIGS. 7A-7D depict portions of a screen illustrating a variety of use cases according to embodiments of the present disclosure.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Systems, devices, and associated methods to quantify double vision and track its progress with treatment of the underlying diagnosis are described herein. The double vision tracking device can utilize an algorithm of tasks to determine if the double vision occurs when one or both eyes are open, which direction is the patient looking at when they experience the double vision, and how many degrees from primary gaze (midline) is the double vision first noted. The double vision tracking device can implement a blind spot calibration procedure to determine the patient's blind spot. Then, the user can be instructed to focus on a fixation marker that moves across a display screen. The user can further be instructed to press a keyboard key when that fixation marker appears double. If the fixation marker appears double, the user is asked if the duplicated objects appear on top of each other (vertical diplopia) or next to each other (horizontal diplopia). The tracking device can compare the location of the object on the screen when the user indicates that the appears double to the location of the user's blind spot and can determine the degrees from midline gaze at which the double vision occurs. As users repeat this test, the degrees at which double vision occurs can be compared to prior tests to determine disease progression or resolution.

In some cases, 4 fixation markers are tested on each patient, but the number of fixation markers can increase depending on the user answers. The 4 locations of the fixation markers are left and right sides of the screen and top and bottom mid-screen (see Figure). The user is asked to turn their head and look straight at the first fixation marker that appears. The marker can be repositioned across the screen (e.g., the left-sided marker moves towards the right, the right-sided towards the left, the top moves down, and the bottom one moves up). The user focuses on the moving marker and presses a keyboard key if that marker appears double. If the user presses a key, the location on the screen and the distance of the marker from the blind spot when the key is pressed is determined in pixels and then converted to degrees.

The size, shape, and color of the markers displayed throughout the screen can be modified based on the patient's medical history as well as prior assessment results. Once the user identifies an area of double vision, more markers can be displayed in that area to further elucidate the boundaries of the area of interest. If the fixation markers do not detect double vision, additional fixation markers may be displayed, for example from each corner of the screen and move diagonally across the screen. The user is asked once again to press a key if the marker appears double at any point.

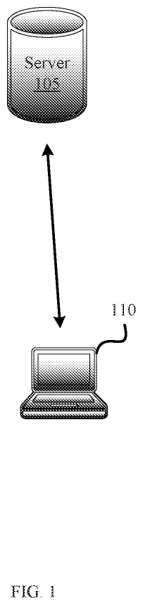

FIG. 1 depicts a system for double vision tracking according to an embodiment of the present disclosure. The system can include a server 105 and a computing device 110.

The server 105 can store instructions for performing a double vision procedure. In some cases, the server 105 can also include a set of processors that execute the set of instructions. Further, the server 105 can be any type of server capable of storing and/or executing instructions, for example, an application server, a web server, a proxy server, a file transfer protocol (FTP) server, and the like. In some cases, the server 105 can be a part of a cloud computing architecture, such as a Software as a Service (SaaS), Development as a Service (DaaS), Data as a Service (DaaS), Platform as a Service (PaaS), and Infrastructure as a Service (IaaS).

A computing device 110 can be in electronic communication with the server 105 and can display the double vision procedure to a user. The computing device 110 can include a display for displaying the double vision procedure, and a user input device, such as a mouse, keyboard, or touchpad, for logging and transmitting user input corresponding to the double vision procedure. In some cases, the computing device 110 can include a set of processors for executing the double vision procedure (e.g., from instructions stored in memory). Examples of a computing device include, but are not limited to, a personal computer, a laptop, a tablet, a cellphone, a personal digital assistant, an e-reader, a mobile gaming device, and the like.

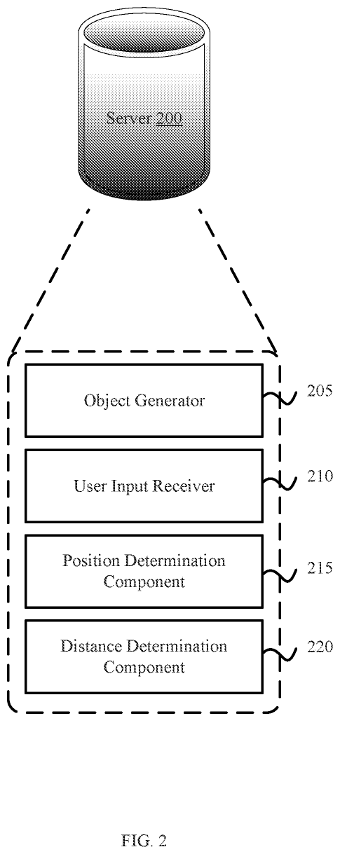

FIG. 2 depicts a server 200 for testing double vision according to an embodiment of the present disclosure. The server can be an example of the server 105 as discussed with reference to FIG. 1. The server 200 can include an object generator 205, a user input receiver 210, an object position determination component 215, and a double vision determination component 220.

The object generator 205 can generate a repositionable animated object for the display screen of the computing device of a user, such as the computing device 110 as described with reference to FIG. 1. The repositionable animated object can be any number of objects having a defined body, which includes but is not limited to a dot, a circle, a triangle, a star, a rectangle, an ellipse, and the like. Further, the object generator 205 can reposition the animated object on the display screen over a period of time. For example, the animated object can move in a predefined direction at a predefined speed across the display upon initiation of the double vision procedure. In some cases, the object generator can also generate a reference point to be displayed by the display. The reference point may be a stationary object displayed on the screen. In some cases, the animated object may move in relation to the reference point, for example moving away from, or towards the reference point.

The user input receiver 210 can receive user input from the computing device. For example, the user input can be a mouse click, a keyboard click, a touch on a touchpad, and the like. The user input receiver 210 can receive the user input and log different parameters of the user input. For example, the user input receiver 210 can identify a timestamp of the user input, the type of user input (e.g., mouse click, keyboard click, etc.) and the like. The server 200 may store the user input in memory.

The double vision determination component 215 can determine a location a user experiences double vision. The double vision determination component 215 can determine a position of the animated object based on the received user input. As discussed above, the animated object may be repositioned on the display screen during the double vision procedure. The double vision determination component 215 can determine the position of the animated object at the time the user provides input via the computing device. The determination can be based on a timestamp of the received user input. In some cases, the determination can be based on the predefined speed, the predefined direction, and/or an initiation timestamp corresponding to when the double vision procedure began (e.g., when the animated object initiated movement on the display).

In some cases, the server 200 can repeat the double vision procedure. For example, in some cases the repositionable object can move in a predefined direction (e.g., from left to right on the display screen). After receiving input for the repositionable object, the object generator 205 may repeat the procedure with a second repositionable object. The second repositionable object may be initially located at a different location on the display compared to the first repositionable object, and may travel in a different direction compared to the first repositionable object (e.g., from right to left on the display). In a particular embodiment, the display may display 4 different repositionable objects: a first object beginning on the left-hand side of the display and traveling from left to right; a second object beginning on the right-hand side of the display and traveling right to left; a third object beginning in a top-half of the display and traveling from top to bottom; and a fourth object beginning on the bottom-half of the display and traveling from bottom to top.

In some cases, the server can also repeat the double vision procedure in a smaller portion of the display. For example, if the system receives input corresponding to a particular region of the display, the server may repeat the double vision process in an area of the display immediately surrounding the particular region of the display. In some cases, the parameters of the repositionable object may alter during the second process. For example, the speed of the repositionable may be slower compared to the first repositionable object. This process may be beneficial in determining where the user experiences double vision in a more accurate manner.

In some cases, the server may request additional input from a user after receiving the initial user input. For example, after receiving the initial user input, the computing device may provide additional instructions for the user to indicate whether the double vision of the repositionable object occurs in a vertical (e.g., one over top the other) fashion or in a horizontal (e.g., side by side) fashion.

In some cases, the double vision determination component 220 can determine an angle from center which the double vision is experienced. For example, the system can determine a distance away from the display the user is positioned, such as by performing a blind spot detection procedure. The blind spot detection procedure can reposition an object across the display screen. The user may be instructed to provide user input (e.g., via a mouse, keyboard, and the like) when the repositionable object is located in an area where the user cannot see the object (e.g., while the user is focused on a particular static object of the display screen). The user may also be instructed to provide input once the object reappears in the user's vision. From the length of the blind spot experienced by the user, the system can determine how far away the user is positioned from the screen. Based on the determined distance and the position of the visual segment corresponding to user input, the report generator 220 can determine an angle from center the double vision is experienced.

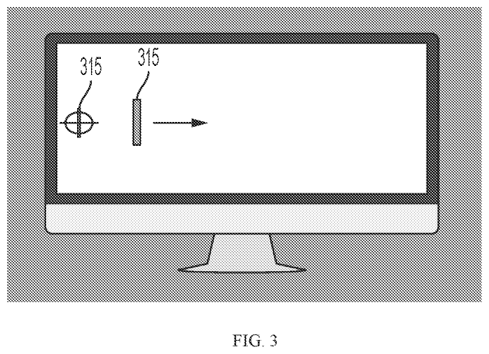

FIG. 3 depicts a screenshot of a double vision procedure according to embodiments of the present disclosure. In FIG. 3, the user may be instructed to focus on a reference point 305, which may be statically positioned. The repositionable object 315 may reposition on the display at a predefined speed and direction. In this case, the repositionable object 315 may move towards and away from the reference point 305. The user is instructed to focus on the reference point 305 while the repositionable object 315 moves away from the reference point 305. The user is further instructed to provide input when the user experiences double vision of the repositionable object 315.

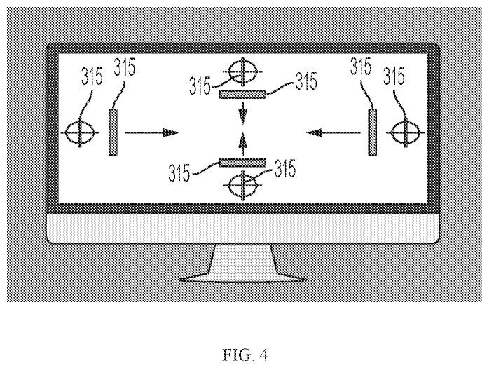

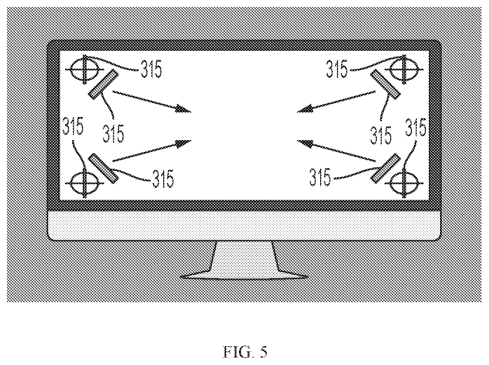

FIG. 4 depicts a screenshot of a double vision procedure according to embodiments of the present disclosure. In this embodiment, the double vision procedure can include four repositionable objects 315-a-d, and corresponding reference points 305-a-d. The sets of reference points/repositionable objects may be displayed at separate time periods (e.g., only one reference point/repositionable object set may be displayed at any given time). The repositionable object 315 can travel away from the respective object 305, and a user is instructed to provide input for when the user views the repositionable object as double. The embodiment depicted in FIG. 4 shows particular starting locations for the repositionable objects and reference points. FIG. 5 depicts an embodiment of the double vision procedure, with the repositionable object and reference points located in different positions compared to the embodiment depicted in FIG. 4.

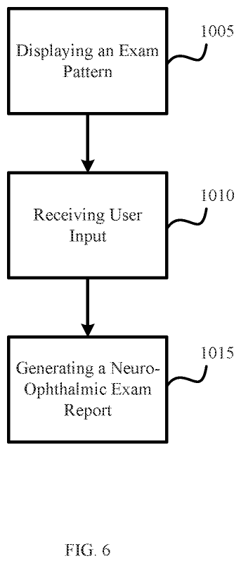

FIG. 6 depicts a process flow for generating a neuro-ophthalmic examination according to an embodiment of the present disclosure. The process flow can be implemented by system 100 of FIG. 1. In some cases, the process flow can be implemented by computing device 110 of FIG. 1.

At Step 605, a marker at a location on a display screen can be displayed. The display can be of a computing device, such as computing device 110 of FIG. 1. In some cases, the marker may be generated by the object generator 205 of FIG. 2.

At Step 610, the marker can be repositioned in different location across the display screen. In some cases, the repositioning can be implemented in a predefined direction and a predefined speed. In some cases, the repositioning can be with respect to a statically positioned reference point displayed on the display.

At Step 615, user input can be received. The user input can be received during a time period which a given segment of the plurality of segments is displayed on the display screen. In some cases, the user input can be received via computer mouse, a touchscreen, a microphone, a keyboard, a video camera, or a combination thereof. The input can be received a user input receiver 210 of FIG. 2.

At Step 620, a given location of the marker when the user input is received can be determined. The location can be determined from a timestamp of the received user input. In some cases, the location can be determined from a predefined speed and direction of the traveling marker. The determination can be made by the position determination component 215 of FIG. 2.

At Step 625, a location where a user experiences double vision can be determined from the received user input. In some cases a degree from a midline gaze can be determined for where the user experiences double vision. In some cases, the double vision can be further determined to be vertical or horizontal double vision. These determinations can be performed by the double vision determination component 220 of FIG. 2.

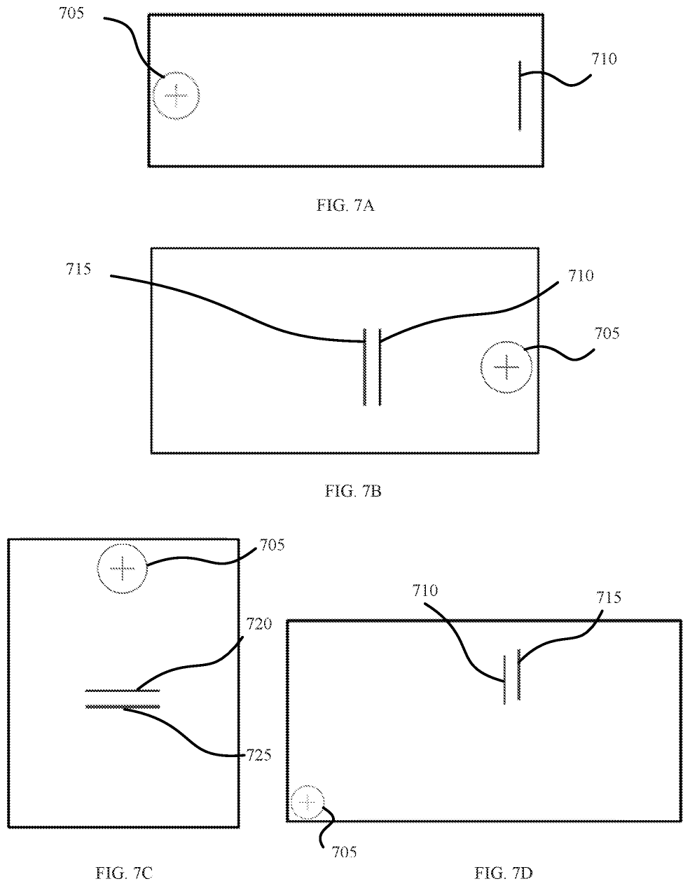

Referring now to FIG. 7A-7D, portions of a screen illustrating a variety of use cases of the present disclosure are illustrated. In accordance with exemplary embodiments of the present disclosure, software for identifying binocular double vision can include a fixation marker at the left, right, top, and bottom of the screen. In certain embodiments, the participant is asked to align their head towards the fixation marker (e.g., fixation marker 705), but focus their eyes on a moving marker (e.g., moving marker 710) that starts from the fixation marker and moves across the screen opposite to the position of the fixation marker. When the participant sees the moving marker as double (e.g., 2 lines instead of 1), the participant presses a keyboard key to indicate at which location the double vision was noted. Based on the software's blind spot calibration, the software calculates the degrees at which double vision is experienced.

In a study of the present disclosure, patients with complaints of double vision were studied. FIG. 7A illustrates a case of a participant with no complaints (or manifestations) of double vision. Such a patient/result can serve as a negative control. FIGS. 7B-7D illustrate three examples of different manifestations of double vision. Double vision can be horizontal or vertical. Patients with horizontal double vision see the duplicated image aside the real image, while patients with vertical double vision see the duplicated image above or below the real image.

Referring specifically to FIG. 7A, “Use Case 1” is illustrated. FIG. 7A illustrates an example of a user with no complaints (or manifestations) of double vision. A vertical marker 710 is illustrated having been moved across the entirety of the screen (e.g., from left to right) without ever being seen as duplicated. Such an example can serve as a negative control for software implementing exemplary embodiments of the present disclosure.

Referring now to FIG. 7B, “Use Case 2” is illustrated. FIG. 7B illustrates an example of a user with complaints (or manifestations) of horizontal double vision who undergo a double vision assessment. The user's head is aligned with fixation marker 705 and the eyes are focused on the vertical marker 710 moving across the screen. The moving vertical marker 710 appears double (i.e., including duplicated vertical marker 715) at the location depicted, at which point the user pressed a keyboard key. Duplicated vertical marker 715 is depicted as a line adjacent to moving vertical marker 710.

Referring now to FIG. 7C, “Use Case 3” is illustrated. FIG. 7C illustrates an example of a user with complaints (or manifestations) of vertical double vision who tests positive when the fixation marker 705 is at the top of the screen and a horizontal marker 720 is moved toward the bottom of the screen. A duplicated horizontal marker 725 is depicted as a line adjacent to moving horizontal marker 720. Based on the user's distance from the screen and their blind spot calibration, the location at which double vision is experienced can be calculated.

Referring now to FIG. 7D, “Use Case 4” is illustrated. FIG. 7D illustrates an example of a user with complaints (or manifestations) of double vision that tested negative on the horizontal and vertical assessments, but tested positive on the diagonal assessment. While the user's head is aligned with fixation marker 705 on the bottom left of the screen, the eyes track the vertical line (i.e., vertical marker 710) that crosses the screen in a diagonal fashion. The moving vertical marker 710 is depicted in the location where the double vision was noted. Duplicated vertical marker 715 is depicted as a line adjacent to moving vertical marker 710. The diagonal assessment can be performed with fixation marker 705 placed in each of the four corners of the screen and vertical marker 710 moving diagonally towards each of the opposite four corners.

### Enumerated Embodiments

The following enumerated embodiments are provided, the numbering of which is not to be construed as designating levels of importance.

Embodiment 1 provides a computer-implemented method including:
displaying a marker at a location on a display screen;
repositioning the marker in different locations across the display screen;
receiving, from a user input mechanism, user input;
determining a given location of the marker when the user input is received; and
determining from the received input, that a user views double vision of the marker at the given location.

Embodiment 2 provides the computer-implemented method of embodiment 1 further including: receiving a second user input indicating whether the double vision is vertical diplopia, horizontal diplopia, or a combination thereof.

Embodiment 3 provides the computer-implemented method of any one of embodiments 1-2, wherein the user input is received via a computer mouse, a touchscreen, a microphone, a keyboard, a video camera, or a combination thereof.

Embodiment 4 provides the computer-implemented method of any one of embodiments 1-3, further including:
determining a blind spot of a user on the display screen; and
determining, from the determined blind spot and the given location the user views double vision, a degree from a midline gaze of the user for the double vision.

Embodiment 5 provides the computer-implemented method of embodiment 4, wherein the determining the blind spot of the user on the display screen further includes:
displaying a repositionable animated object on the display screen;

receiving a second user input when the repositionable animated object transitions from within a viewing range of the user to outside the viewing range;

determining a position of the repositionable animated object on the display screen based on a timing the second user input is received; and

determining a distance away from the display screen for the user based on the position of the repositionable animated object.

Embodiment 6 provides the computer-implemented method of any one of embodiments 1-5, wherein the repositioning of the marker on the display screen is in a left-to-right direction, a right-to-left direction, an up-to-down direction, or a down-to-up direction.

Embodiment 7 provides the computer-implemented method of embodiment 6, further including:
displaying on the display screen a second marker at a second location;
repositioning the second marker in a direction different than a direction for repositioning the marker;
receiving a second user input from the user input mechanism;
determining a second given location of the second marker when the second user input is received; and
determining from the second user input, that the user views double vision of the second marker at the second given location.

Embodiment 8 provides the computer-implemented method of embodiment 7, wherein the marker is removed from the display screen after receiving the user input.

Embodiment 9 provides the computer-implemented method of any one of embodiments 1-8, further including:
displaying a second marker in a subsection of the display screen enveloping the given location;
repositioning the second marker in a second set of different locations within the subsection of the display screen;
receiving a second user input from the user input mechanism;
determining a second given location of the second marker when the second user input is received; and
adjusting the determination that the user views double vision at the given location based on the second given location.

Embodiment 10 provides a device for generating a neuro-ophthalmic examination report, including:
a display screen;
a user input mechanism; and
one or more processors configured to execute a set of instructions that cause the one or more processors to:
   display a marker at a location on the display screen;
   reposition the marker in different locations across the display screen;
   receive, from the user input mechanism, user input;
   determine a given location of the marker when the user input is received; and
   determine, from the received input, that a user views double vision of the marker at the given location.

Embodiment 11 provides a computer-readable medium for generating a neuro-ophthalmic examination report, including:
one or more processors;
memory; and
a set of instructions stored in the memory that, when executed by the one or more processors, cause the one or more processors to:
   display, via a display screen, a marker at a location on the display screen;
   reposition the marker in different locations across the display screen;
   receive, from a user input mechanism, user input;
   determine a given location of the marker when the user input is received; and
   determine, from the received input, that a user views double vision of the marker at the given location.

Embodiment 12 provides the device of embodiment 10, wherein the device is configured and adapted to implement any of the methods of embodiments 1-9.

Embodiment 13 provides the computer-readable medium of embodiment 11, wherein the computer-readable medium is configured and adapted to implement any of the methods of embodiments 1-9.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A computer-implemented method comprising:
displaying a marker at a location on a display screen;
repositioning the marker in different locations across the display screen;
receiving, from a user input mechanism, user input;
determining a given location of the marker when the user input is received; and
determining from the received input, that a user views double vision of the marker at the given location.

2. The computer-implemented method of claim 1, further comprising:
receiving a second user input indicating whether the double vision is vertical diplopia, horizontal diplopia, or a combination thereof.

3. The computer-implemented method of claim 1, wherein the user input is received via a computer mouse, a touchscreen, a microphone, a keyboard, a video camera, or a combination thereof.

4. The computer-implemented method of claim 1, further comprising:
determining a blind spot of a user on the display screen; and
determining, from the determined blind spot and the given location the user views double vision, a degree from a midline gaze of the user for the double vision.

5. The computer-implemented method of claim 4, wherein the determining the blind spot of the user on the display screen further comprises:
displaying a repositionable animated object on the display screen;
receiving a second user input when the repositionable animated object transitions from within a viewing range of the user to outside the viewing range;
determining a position of the repositionable animated object on the display screen based on a timing the second user input is received; and
determining a distance away from the display screen for the user based on the position of the repositionable animated object.

6. The computer-implemented method of claim 1, wherein the repositioning of the marker on the display screen is in a left-to-right direction, a right-to-left direction, an up-to-down direction, or a down-to-up direction.

7. The computer-implemented method of claim 6, further comprising:
displaying on the display screen a second marker at a second location;
repositioning the second marker in a direction different than a direction for repositioning the marker;
receiving a second user input from the user input mechanism;
determining a second given location of the second marker when the second user input is received; and
determining from the second user input, that the user views double vision of the second marker at the second given location.

8. The computer-implemented method of claim 7, wherein the marker is removed from the display screen after receiving the user input.

9. The computer-implemented method of claim 1, further comprising;
displaying a second marker in a subsection of the display screen enveloping the given location;
repositioning the second marker in a second set of different locations within the subsection of the display screen;
receiving a second user input from the user input mechanism;
determining a second given location of the second marker when the second user input is received; and
adjusting the determination that the user views double vision at the given location based on the second given location.

10. A device for generating a neuro-ophthalmic examination report, comprising:
a display screen;
a user input mechanism; and
one or more processors configured to execute a set of instructions that cause the one or more processors to:
display a marker at a location on the display screen;
reposition the marker in different locations across the display screen;
receive, from the user input mechanism, user input;
determine a given location of the marker when the user input is received; and
determine, from the received input, that a user views double vision of the marker at the given location.

11. A computer-readable medium for generating a neuro-ophthalmic examination report, comprising:
one or more processors;
memory; and
a set of instructions stored in the memory that, when executed by the one or more processors, cause the one or more processors to:
display, via a display screen, a marker at a location on the display screen;
reposition the marker in different locations across the display screen;
receive, from a user input mechanism, user input;
determine a given location of the marker when the user input is received; and
determine, from the received input, that a user views double vision of the marker at the given location.
